SEQUENCE LISTING (15 sequences)

SEQ ID NO: 1 - DNA, 21 bp, Artificial Sequence
  primer
ggctgagctgggtggtcctgg

SEQ ID NO: 2 - DNA, 21 bp, Artificial Sequence
  primer
ggctgagtttggtggtcctgg

SEQ ID NO: 3 - DNA, 23 bp, Artificial Sequence
  primer
ggtacgtgctgttgaactgttcc

SEQ ID NO: 4 - DNA, 55 bp, Artificial Sequence
  primer
catttgagttggcctagccggccatggcagaggtgcagctggtggagtctggggg

SEQ ID NO: 5 - DNA, 35 bp, Artificial Sequence
  primer
catgggcccagccggccgagctggcctcagcacat

SEQ ID NO: 6 - DNA, 30 bp, Artificial Sequence
  primer
catggcggccgcggggtgtacatgaaatcg

SEQ ID NO: 7 - DNA, 33 bp, Artificial Sequence
  primer
cagtgcggccgccgaagtcaccattgaggctgt

SEQ ID NO: 8 - DNA, 29 bp, Artificial Sequence
  primer
actggttaacggggtgtacatgaaatcgc

SEQ ID NO: 9 - DNA, 27 bp, Artificial Sequence
  primer
gcggccgccgaggtgcagctggtggag

SEQ ID NO: 10 - DNA, 26 bp, Artificial Sequence
  primer
gcggccgctgaggagacggtgacctg

SEQ ID NO: 11 - DNA, 19 bp, Artificial Sequence
  primer
ccagtaagcaataatgtgg

SEQ ID NO: 12 - DNA, 22 bp, Artificial Sequence
  primer
gacttagtcctctccttgattg

SEQ ID NO: 13 - DNA, 18 bp, Artificial Sequence
  primer
cttcaacggtctcagtgc

SEQ ID NO: 14 - protein, 120 aa, Artificial Sequence
  nanobody sequence
EVQLVESGGGLVQAGGSLRLSCAASGITFRRYTMGWYRQAPGKQRELVASINSSDTTNYADSVKGRFTISRDNAKNTVYLQMNSLKPEDTAVYYCNARWCISQFVDNTYWGQGTQVTVSS

SEQ ID NO: 15 - protein, 120 aa, Artificial Sequence
  nanobody sequence
EVQLVESGGGLVQAGGSLRLSCATSGITFMRYALGWYRQSPGKQREMVASINSGGTTNYADSVKGRFTISRDNAKNTVYLQMNSLKPEDTAVYYCNARWVKPQFIDNNYWGQGTQVTVSS

(19) United States
(12) Patent Application Publication
(10) Pub. No.: US 2016/0168258 A1
(43) Pub. Date: Jun. 16, 2016

(54) BI-SPECIFIC ADAPTERS

(71) Applicants: Hélène VERHEIJE, CM Utrecht (NL); Paul VAN BERGEN EN HENEGOUWEN, CM Utrecht (NL); Peter ROTTIER, CM Utrecht (NL); Marta KIJANKA, CM Utrecht (NL)

(72) Inventors: Helene Verheije, Utrecht (NL); Paul van Bergen en Henegouwen, Utrecht (NL); Peter Rottier, Utrecht (NL); Marta Kijanka, Utrecht (NL)

(21) Appl. No.: 14/384,404

(22) PCT Filed: Mar. 12, 2013
(86) PCT No.: PCT/EP2013/054940

§ 371 Date: Sep. 11, 2014

(30) Foreign Application Priority Data
Mar. 13, 2012 (EP) 12159322.2

(51) Int. Cl. C07K 16/30; C12N 7/00; C07K 14/005

(52) U.S. Cl. CPC C07K 16/30; C07K 14/005; C12N 7/00; C07K 2317/569; A61K 39/00

(57) ABSTRACT

The present invention relates to bi-specific adapters for re-directing viruses to non-virus specific host cells, to expression cassettes comprising a DNA molecule having a nucleotide sequence encoding such bi-specific adapters, to recombinant Coronaviruses comprising such expression cassettes and to their use as a medicament and their use in the treatment of tumors.

The present invention relates to bi-specific adapters for re-directing viruses to non-virus specific host cells, to expression cassettes comprising a DNA molecule having a nucleotide sequence encoding such bi-specific adapters, to recombinant Coronaviruses comprising such expression cassettes and to their use as a medicament and their use in the treatment of tumors.

Already for quite some years oncolytic viruses are being investigated for use in tumor therapy (for recent reviews, see references [1, 2, 3, 4, 5]). Their success in destroying cancer cells depends on their ability to selectively infect and kill these cells. Although some oncolytic viruses appear to have a natural tropism for tumor cells, most viruses need to be modified in some way to achieve infection and/or lytic activity in these cells. One of the ways to accomplish specific infection of tumor cells is by redirecting the virus to epitopes expressed on such cells. Thus, different targeting approaches have been explored for a variety of viruses. These include pseudo typing, modification of viral surface proteins, and the use of bi-specific adapters (vide infra and [6, 7, 8]. All of these approaches require that the viability of the virus is not hampered and that the targeting moiety is properly exposed to allow directed infection. The ability to genetically modify a particular virus combined with the availability of an appropriate targeting epitope determines the success of the approach.

So far, there are only a few reports describing the potential use of Coronavirus-based oncolytic agents in cancer therapy. Coronaviruses are positive-strand RNA viruses consisting of a nucleocapsid, which contains the approximately 30 kb genome and the nucleocapsid (N) protein, and which is surrounded by an envelope carrying three membrane proteins, spike (S), envelope (E), and matrix (M). Of these, the spike glycoprotein S is responsible for virus entry and syncytia formation, as it binds to the cellular receptor and induces membrane fusion[9, 10, 11].

Most Coronaviruses exhibit strict species specificity, as determined by the spike-receptor interaction[12, 13, 14].

The Coronavirus feline infectious peritonitis virus (FIPV), for instance, selectively infects and induces syncytium formation in feline cells via its receptor feline aminopeptidase N (fAPN). [15]. Likewise, the recombinant felinized mouse hepatitis virus (fMHV), [16] a derivative of mouse hepatitis virus (MHV) carrying a chimeric spike of which the ectodomain is of the FIPV spike protein, also infects and fuses only feline cells through the fAPN molecule. As a consequence of their species restricted tropism, FIPV and MHV are nonpathogenic to non-feline cells or non-murine cells respectively. However, once the tropism barrier is alleviated, Coronaviruses can replicate in cells of different species [16, 17]. Thus, FIPV and MHV may potentially be converted into specific oncolytic agents for the treatment of cancer if their spike protein would recognize a receptor on tumor cells.

The non-human Coronavirus murine hepatitis virus (MHV) is the best-studied Coronavirus and more importantly, for Coronaviruses in general, convenient reverse genetics systems are available to modify the Coronaviral genome [16, 18].

MHV, as several other Coronaviruses, has several appealing characteristics that might make it suitable as an oncolytic virus.

First, it has a narrow host range, determined by the interaction of its spike (S) glycoprotein with the cellular receptor mCEACAM1a [19].

Since mCEACAM1a is not expressed on non-murine cells, MHV cannot establish an infection in either normal or cancerous non-murine cells.

Second, infection by MHV induces the formation of large multinucleated syncytia, which means: fusion of the infected cell with surrounding uninfected cells [11]. Hence, given also its relatively short replication cycle (6 to 9 h), MHV destroys populations of cells rapidly once they have become infected.

Third, the tropism of MHV can be modified either by substitution of the viral spike ectodomain or by the use of bi-specific adapters [20, 21, 22, 23].

These bi-specific adapters are proteins comprising a virus-binding moiety and a target cell-binding moiety. Such proteins on the one hand specifically bind to a Coronavirus and on the other hand they specifically bind to a specific receptor on a target cell. Therefore, they act as an intermediate between a Coronavirus and a target cell, and as such they are able to redirect a specific Coronavirus to a specific target cell that normally would not be infected by that Coronavirus. Studies performed with such bi-specific adapters revealed that, once the host cell tropism barrier is alleviated, e.g. MHV is capable of establishing infection in non-murine cells.

For Coronaviruses, such bi-specific adapters have i.a. been described by Wurdinger [22]. This paper describes the use of a bi-specific single-chain antibody as a bi-specific adapter for targeting non-human Coronaviruses to human cancer cells. The virus-binding moiety used in this paper originates from an antibody raised against the FIP Spike protein whereas the target cell-binding moiety originates from an antibody raised against the Human Epidermal Growth Factor Receptor (EGFR).

In a further paper by Wurdinger [21], another bi-specific adapter is described that comprises the N-terminal part of the MHV cellular receptor CEACAM1a, the so-called soluble receptor (soR) (the N-terminal domain of the part of the receptor that protrudes from the cell surface), as the MHV-binding moiety and an antibody raised against the Human Epidermal Growth Factor receptor (EGFR) as the target cell-binding moiety.

However, the use of such bi-specific adapters to target viruses to (tumor) cells has two disadvantages; 1) the bi-specific adapter has to be provided separately and has to be administered to the host together with the virus, preferably bound to the virus and 2) it needs to be re-administered to a host each time the virus has finished a replication cycle and yields new virus particles. This is necessary to redirect de novo made virus particles to infect further (tumor) cells.

Theoretically, in order to overcome these obstacles, genetic information encoding a bi-specific adapter could be introduced into the viral genome to allow the virus to produce the adaptor itself in infected cells, thereby creating self-targeting progeny virus.

The theoretical feasibility of this concept was proven by inserting an expression cassette encoding a bi-specific adapter, composed of soR and a His tag, in the MHV genome [20]. This self-targeting recombinant Coronavirus was shown to be able to infect recombinant human cells expressing an artificial His tag receptor.

In [23], it was shown that a comparable recombinant MHV virus, however now encoding a bi-specific adapter composed of the soR and the Human Epidermal Growth Factor (EGF) was capable of multi-round infection in EGF receptor-expressing cells, resulting in extensive cell-cell fusion and efficient killing of target glioblastoma cells. Using an orthotopic intracranial tumor model of aggressive U87ΔEGFR glioblastomas in nude mice, Verheije [23] showed for the first time that redirected recombinant Coronavirus indeed has oncolytic potential.

However, such an approach has a very limited applicability for the following reason;
1) The Coronavirus genome has by nature a very restricted tolerance with regard to the characteristics of the expression cassettes to be inserted. An expression cassette encoding soR and either the His tag of 18 nucleic acids or EGF of 159 nucleic acids can be inserted in the Corona viral genome, but an expression cassette encoding e.g. soR and a single chain antibody is not tolerated [20,21].
2) The approach is not a versatile approach, for the reason under 1) and due to the fact that for every oncolytic application of the virus a new target cell-binding moiety has to be found, developed and cloned, and usually the size and/or gene characteristics of such a new target cell-binding moiety will exceed the insertion tolerance of the Coronavirus genome.

Therefore, there is a need for more versatile oncolytic recombinant Coronaviruses.

It is an objective of the present invention to provide such oncolytic recombinant Coronaviruses.

It was surprisingly found now that in spite of the very restricted tolerance of the Coronavirus genome for foreign sequences, a bi-specific adapter that comprises a Coronavirus binding moiety and a camelid VHH antibody can successfully be expressed in a Coronavirus.

As used herein, a “bi-specific adapter that comprises a Coronavirus binding moiety and a camelid VHH antibody moiety” is to be understood as follows: such an adapter is a protein that is capable of binding with one side to a Coronavirus (this is the Coronavirus binding moiety) and with another side to a cellular component, whereby the binding of said another side to said cellular component is effected because said another side comprises a so-called camelid VHH antibody directed against said cellular component (this is the camelid VHH antibody moiety).

In particular the bi-specific adapter according to the invention is a protein wherein the Coronavirus binding moiety is located at the N-terminal side of the VHH antibody moiety, or wherein the Coronavirus binding moiety is located at the C-terminal side of the VHH antibody moiety. There is no absolute need for the Coronavirus binding moiety or the VHH antibody moiety to be at the C-terminal or N-terminal end of the bi-specific adapter.

One of the important advantages of such a bi-specific adapter is that, due to the use of a camelid VHH antibody moiety, it can easily be tailored towards binding with each and every tumor-specific protein. This turns such bi-specific adaptors into very versatile instruments for the targeting of Coronaviruses to tumor cells.

Another important advantage of such a bi-specific adapter is, that a cassette expressing such an adapter will always be tolerated by a Coronavirus. This is i.a. due to the fact that this cassette always has practically the same (small) size, because the part of the cassette encoding the camelid VHH antibody moiety always has practically the same size. It is also due to the fact that the VHH protein always has the same basic structure; the differences in amino acid sequence between VHH antibodies raised against one protein or another protein are relatively marginal.

And the main advantage is that insertion of such an expression cassette into a Coronavirus allows the virus to produce the adaptor itself in infected cells, thereby creating self-targeting progeny virus.

Thus, a first embodiment of the present invention relates to a bi-specific adapter, characterised in that said bi-specific adapter comprises a Coronavirus binding moiety and a camelid VHH antibody moiety.

Camelid VHH antibodies are well-known in the art for over two decades already. They are currently also frequently referred to as Nanobodies®. VHH antibodies are defined as the variable region of the heavy chain only antibodies that are present in the family of camelidae, among which is Llama glama. The existence of heavy chain-only antibodies was discovered more than 20 years ago and since then the application of the variable region from these antibodies has been developed in different directions.

The use of such Nanobodies in bi-specific adapters for specific targeting of viruses to cells was unknown, let alone that the incorporation of expression cassettes expressing such bi-specific adapter, in viruses has been suggested.

Camelid VHH antibodies suitable for use as a camelid VHH antibody moiety in a bi-specific adaptor according to the invention are easily induced through immunization of a camelid such as a dromedary or llama with cell surface proteins of a target cell. Such a target cell can be a tumor cell. And in that case, a cell surface protein would function as a tumor specific cell surface protein. A tumor-specific antigen is an antigen produced by a particular type of tumor and that does not or in much lesser amounts appear on normal cells of the tissue from which the tumor developed. Many human tumor-specific antigens are known in the art, such as receptors that belong to the family of growth factor receptors, e.g. Erb, which includes the Epidermal Growth Factor Receptor (EGRF) and Human Epidermal Growth Factor Receptor 2 (HER2). While EGFR is overexpressed in 60-70% of all tumors, Her2 is a specific marker for breast cancer.

Examples of other tumor specific antigens are Carcinoembryonic antigen (CEA), cell surface associated Mucin 1 (MUC-1), epithelial tumor antigen (ETA), Hepatocyte Growth Factor Receptor, IGF-like Growth Factor Receptor 1(IGF-1R), Vascular Endothelial Growth Factor (VEGF), carbonic anhydrase IX (CA-IX) and Glucose Transporter 1 (Glut1).

Examples of tumor-specific antigens found in dogs are e.g. the skin cancer specific protein Ki67, mammary cancer specific c-kit proto-oncogene (PDGF receptor), type IX collagen and the lymphoma-specific protein AgNOR and receptors from the ErbB family (EGFR and Her2). From these tumor markers, the Her2 receptor is frequently (over)expressed in dog osteosarcoma.

The subsequent isolation and cloning of the repertoire of antigen-binding fragments from an immunized animal into a phage display vector and the selection of antigen-specific clones by panning has become a routine method for selecting antigen-specific molecules for well over a decade already [24, 25].

This method has been adapted to Nanobodies, whereby the single-chain nature of Nanobodies simplifies the method considerably [26]. Tapping the antigen-binding repertoire of the heavy chain antibodies of an immunized dromedary or llama is less complicated with respect to the repertoire cloning of conventional antibodies, for example, in the form of single chain variable fragments (scFv), as the intact antigen-binding site is encoded by a single gene fragment.

Generally, cDNA is prepared from peripheral blood lymphocytes, isolated from an immunized dromedary or llama. As all Nanobodies belong to one single gene family, they are encoded by a single exon with homologous border sequences. Consequently, the complete in vivo matured Nanobody repertoire of a single immunized animal can be amplified by a single set of primers. A secondary polymerase chain reaction with nested primers is then performed to produce more material and to include restriction enzyme sites for cloning purposes. Following cloning of the amplified Nanobody gene fragments in the appropriate expression vector, a Nanobody library containing the repertoire of the intact in vivo matured antigen-binding sites is obtained [26]. Because of the in vivo maturation of VHH's, libraries of about 10⁷ to 10⁸ individual Nanobody genes have routinely resulted in the isolation of Nanobodies with nanomolar affinity for their antigen[26, 27, 28].

Nanobody libraries can be screened for the presence of antigen-specific binders either by direct colony screening or by panning. Retrieval of binders by panning is the preferred method, as panning allows selection for binders with the highest affinities [29].

An example of the cloning of the DNA encoding the desired Nanobody into an expression cassette encoding the bi-specific adaptor, and the introduction of such an expression cassette into a Coronavirus genome is shown in the Examples section below.

Nanobodies, their use and ways of producing them have been described i.a. in reviews in [30-33].

For human use, Nanobodies can, if desired, be humanised as i.a. described in [34].

With regard to the coronavirus binding moiety of the bi-specific adaptor, it is highly advantageous to select a Coronavirus Spike protein receptor as the coronavirus binding moiety. If MHV is the oncolytic virus of choice, the Spike protein cellular receptor CEACAM1a would be the cellular receptor of choice. CEACAM1a has been described above (vide supra).

For several other Coronaviruses, the Coronavirus Spike protein receptor is also known. For Transmissible Gastroenteritis virus (TGEV), the cellular receptor is the Porcine Aminopeptidase N [35, 36]. For Feline Infectious Peritoneitis virus (FIP), it is the Feline Aminopeptidase N [37]. Interestingly, the FIP Spike protein cellular receptor binds to several Group I Coronaviruses such as Human Coronavirus HCV-229E and to TGEV, and can therefore be used as a more universal receptor for Group I Coronaviruses.

Thus, a preferred form of this embodiment relates to a bi-specific adapter according to the invention, wherein the Coronavirus binding moiety comprises a Coronavirus Spike-protein receptor.

In principle, the N-terminal part of CEACAM1a, the so-called soluble receptor (soR), a domain of the part of the receptor that protrudes from the cell surface, or alternatively the spike-binding domain of Porcine Aminopeptidase N or Feline Aminopeptidase N would be preferred as the Coronavirus-binding moiety. The use of only the soluble part of the receptor ensures that the capability to bind to the Spike protein is maintained, while at the same time the risk of incorrect expression and processing of the bi-specific adapter due to the presence of hydrophobic regions is eliminated.

Therefore, a more preferred form of this embodiment relates to a bi-specific adapter according to the invention, characterised in that said Coronavirus binding moiety only comprises a soluble part of the Coronavirus Spike-protein receptor.

If a Coronavirus Spike-protein receptor is selected as the Coronavirus binding moiety, then preferably the Coronavirus Spike-protein receptor is an MHV Spike-protein receptor, a FIP Spike-protein receptor or a TGEV Spike-protein receptor.

Therefore an even more preferred form of this embodiment relates to a bi-specific adapter according to the invention wherein said Coronavirus Spike-protein receptor is selected from the group consisting of MHV Spike-protein receptor, FIP Spike-protein receptor and TGEV Spike-protein receptor, in particular the soluble part of these receptors.

As indicated above, one of the most important uses of bi-specific adapters according to the invention is in targeting the host cell specificity of oncolytic Coronaviruses to tumor cells. Thus, a still even more preferred form of this embodiment relates to a bi-specific adapter according to the invention wherein said camelid VHH antibody moiety is directed against a tumor-specific antigen.

As mentioned above, the bi-specific adaptor can e.g. be made by expressing a nucleotide sequence that comprises the genetic code for the bi-specific adapter. This nucleotide sequence preferably additionally comprises regulatory sequences that affect/influence the expression of the bi-specific adapter.

The nucleotide sequence encoding a bi-specific adapter according to the invention is preferably placed under the control of a functional transcription regulatory sequence (TRS). In positive-stranded RNA (Corona-) viruses, transcription regulatory sequences function as the equivalent of cellular promoters to regulate the expression of downstream genes in the viral genome.

If the expression cassette according to the invention is integrated in the viral genome, it will usually comprise a transcription regulation sequence (TRS) and/or be integrated downstream a TRS. Preferably, this is a Coronaviral TRS. Examples of such TRS and suitable insertion sites are given in i.a. de Haan (2002) and (2003) [48, 51].

Thus, another embodiment of the present invention relates to an expression cassette comprising an RNA or DNA molecule comprising a nucleotide sequence that encodes a bi-specific adapter according to the invention, under the control of a TRS. In the virus, the expression cassette would be in the form of RNA, but during the cloning phase of the expression cassette, the cassette would be in the form of DNA. This is illustrated in the Examples section (vide infra).

An expression cassette is understood to be a stretch of RNA or DNA that comprises genetic information encoding a bi-specific adapter according to the invention under the control of a TRS.

As said above, it was surprisingly found that in spite of the very restricted tolerance of the Coronavirus genome for inserted expression cassettes, a recombinant Coronavirus comprising an expression cassette encoding a bi-specific adapter that comprises a Coronavirus binding moiety and a camelid VHH antibody moiety is still viable.

Therefore, another embodiment of the present invention relates to recombinant Coronaviruses, characterised in that said recombinant Coronaviruses comprise an expression cassette according to the invention.

Still another embodiment of the present invention relates to recombinant Coronaviruses according to the invention, for use as a medicament.

A preferred form of this embodiment relates to recombinant Coronaviruses according to the invention, for use in the treatment (eradication) of a tumor.

With regard to the use of recombinant Coronaviruses according to the invention, the following can be said: the viruses are administered in a live form and already carrying the adapter bound to their spikes, so immediately after administration the viruses will target to tumor cells displaying the specific tumor specific antigen to which the VHH has been raised.

And since recombinant Coronaviruses according to the invention are capable of producing the adaptor itself in infected cells, they create self-targeting progeny virus. This progeny virus in turn can infect new tumor cells. Therefore even a low amount of virus particles is capable of eventually clearing a high number of tumor cells.

Therefore, amounts as low as 10³ viruses can in principle be expected to eventually clear a tumor.

However, since recombinant Coronaviruses according to the invention only bind to cells displaying a tumor specific protein, they are basically non-toxic for non-tumor cells. Therefore, higher doses up to 10⁸ virus particles can in principle be applied without adverse effects.

With regard to the amount of virus particles administered, it should be kept in mind that the host's immune system recognizes the virus as foreign and will respond by raising an immune reaction. The induction of an immune response occurs at a certain speed. Consequently, a disadvantage of administering low amounts of virus particles is, that it may take several rounds of replication before the virus titer in the host is sufficiently high to attack all tumor cells. And during this period, the immune system matures and starts neutralising the virus. This problem can easily be avoided by administering larger amounts of virus. It will then consequently take less rounds of replication before the virus titer in the host is sufficiently high to attack all tumor cells.

In such cases, a dose between 10⁵ and 10⁸ virus particles may be the preferred dose.

An elegant alternative to escape the effects of the induction of immunity is the following: a first attack can be made with a first Coronavirus according to the invention. As soon as the immune response towards this virus reaches the level that leads to removal of the Coronavirus, a second Coronavirus according to the invention, targeted against the same cell (and preferably against the same specific receptor) can be used for a second attack. Provided that this second Coronavirus has no significantly immunological cross-reaction with the first Coronavirus, the second Coronavirus would not be hampered by the immunological reaction induced against the first Coronavirus. Merely as an example: in this approach, the first Coronavirus according to the invention can be MHV, and the second Coronavirus according to the invention can be FIPV.

If there are reasons to believe that the tumor specific antigen is not fully tumor-specific and therefore may also be present on non-tumor cells albeit in much lower amounts, it would be safe to administer a lower dose of virus particles. In such cases, a dose between 10⁴ and 10⁶ virus particles may be the preferred dose.

Administration of the recombinant Coronavirus according to the invention is preferably done through injection. The virus is preferably administered in a pharmaceutically acceptable solution such as a physiological salt solution or a buffer.

The virus may e.g. be administered directly into the blood stream as tumors are typically well-perfused. For the treatment of solid tumors it may however also be advantageous to administer the virus in or around the tumor. Depending upon the amount of virus administered, multiple doses at multiple sites and/or moments in time may be required.

Again another embodiment of the present invention relates to a pharmaceutical composition comprising a bi-specific adapter according to the invention.

Still another embodiment of the present invention relates to a pharmaceutical composition comprising an expression cassette according to the invention.

And still another embodiment of the present invention relates to a pharmaceutical composition comprising a recombinant Coronavirus according to the invention.

The Examples below provide ample information about how to administer a recombinant Coronavirus according to the invention in an in vivo situation.

### LEGEND TO THE FIGURES

FIG. 1: (A) Schematic representation of a conventional (left) and heavy-chain only antibody (middle). CH, VH: constant and variable domain of heavy chain; CHH, VHH: constant and variable domain of heavy chain from heavy-chain-only antibodies; (B) Amino acid sequence of HER2-binding VHH's 11A4 en 1C8.

FIG. 2: Schematic representation of the soR-based targeting constructs. soR: N-terminal domain of mCEACAM1a; Igκ: signal sequence; myc: myc tag; His: 6-histidine residue tag; Ala: 3-alanine residue tag; VHH: variable domain of heavy chain from heavy-chain-only antibodies sequence; T7: T7 promoter

FIG. 3: Targeting of MHV using VHH-based adapter proteins to human ovarian cancer cells. Adapter proteins produced in a vaccinia T7-based expression system were incubated with MHV and subsequently used to inoculate (A) control CHO-scFv.His, (B) human ovarian MCF7, and (C) human ovarian SKOV3 cells. At 20 h post infection the cells were fixed and stained with an antibody directed against MHV.

### EXAMPLES

### Example 1

### Immunisation of Llama Glama with MCF7 Cells

In order to induce a humoral immune response directed towards the cell surface proteins of human ovarian carcinoma cells, llamas were injected with intact human cell preparations of MCF7 cells (approximately 10⁸ cells per injection). Each animal received seven doses of subcutaneously administered antigen at weekly intervals. Pre-immune and immune sera were collected at days 0 (before immunisation), and after 4 and 6 weeks of immunisation. Four days after the last antigen injection, blood was collected, and periferal blood lymphocytes (PBLs) were purified by density gradient centrifugation on Ficoll-Paque™ PLUS gradients (Amersham Biosciences, Little Chalfont, UK), resulting in the isolation of approximately 10⁸ PBLs.

### Construction of Phage VHH Repertoires

Total RNA was extracted from these PLBs as described (50) and transcribed into cDNA using an oligo-dT primer and the SuperScript III First-Strand Synthesis System for RT-PCR (Invitrogen, Carlsbad, Calif., USA) according to the protocol of the manufacturer. Next, cDNA was treated with RNAse H to deplete for residual RNA prior to purification with the QIAquick PCR Purification Kit (Qiagen, Venlo, The Netherlands). The purified cDNA was then used as template to amplify the repertoire of Ig heavy chain-encoding gene segments with the use of two forward framework 1 (FR1) specific primers 5′-GGCTGAGCTGGGTGGTCCTGG-3′ and 5′-GGCTGAGTTTGGTGGTCCTGG-3′ in 4:1 ratio and a reverse CH2 fragment primer 5′-GGTACGTGCTGTTGAACTGTTCC-3′. This amplification procedure resulted in PCR fragments of approximately 700 bp (representing heavy chain only antibodies from FR1 to CH2) and fragments of 900 bp (representing conventional antibodies from FR1 to CH2). The two classes of heavy chain-encoding genes were then size-separated on agarose gels and genes encoding heavy-chain only IgG were purified with QIAquick PCR Purification Kit (Qiagen, Venlo, The Netherlands). In the next step purified DNA was used as a template in nested PCR, in which a SfiI site was introduced at the 5′ end of the heavy chain only antibody fragment by a

forward FR1 specific primer
5′-CATTTGAGTTGGCCTAGCCGGCCATGGCAGAGGTGCAGCTGGTGGA
GTCTGGGGG-3′.

Since a BstEII restriction site naturally occurs in approximately 90% of the FR4 of VHH genes, the repertoire of PCR-amplified genes was cut with SfiI and BstEII and the resulting 400 bp cDNA fragments were purified by gel electrophoresis. cDNA fragments were finally ligated in phagemid vector pUR8100 for display on filamentous bacteriophage (52) and electro-transformed to Escherichia coli TG1 (K12, Δ(lac-pro), supE, thi, hsdD5/F′traD36, proA+B+, lacIq, lacZΔM15). This resulted in ‘immune’ VHH repertoires of approximately 10⁶ transformants each.

### Phage Display Selection of Anti HER2 VHH Fragments

To select antibodies that bind to the human HER2 receptor several phage display selections were performed. In a first approach anti HER2 phages were selected on captured recombinant purified HER2 ectodomain (ECD) (R&D Systems, Oxon, UK). Maxisorp plates (Nunc, Rochester, Minn., USA) were coated overnight at 4° C. with polyclonal rabbit anti human IgG antibody at dilution 1/500 (DakoCytomation, Glostrup, Denmark), then the plate was washed 3 times with PBS and incubated with decreasing amounts of recombinant purified HER2-ECD in PBS (0.5 μg/well; 0.1 μg/well; 0.05 μg/well; 0.01 μg/well) for 2 h at RT. The non bound HER2-ECD was washed away with PBS and the coated wells were blocked with 4% milk powder in PBS for 1 h at RT. Phages prepared from the ‘immune’ libraries and preblocked with 4% milk powder for 30 min at RT at head-over-head were then panned for binding to immobilized HER2-ECD. After extensive washing with PBS/0.05% Tween-20, phages were eluted with 1 mg/ml trypsin (Sigma-Aldrich), in PBS for 30 min, then trypsin was neutralized by addition of 2 mg/ml trypsin inhibitor in MilliQ (Sigma-Aldrich). Displaced phages were used to infect exponentially growing E. coli TG1 for 30 min at 37° C. Bacteria were plated on LB agar plates containing 2% (w/v) glucose and 100 μg/ml ampicillin. In this set up VHH-phage 1C8 was selected based on its ability to bind the HER2-ECD ectodomain with high affinity.

In a second approach, phages, prepared from ‘immune’ libraries, were panned in two steps: on live BT474 cells in solution in the first round and on biotynylated HER2-ECD in the second round. Briefly, phages were incubated with differing amounts of BT474 cells (from 4*10⁵ cells to 4*10³ cells) in HybriCare Medium (with fetal calf serum, penicillin, streptomycin and glutamine) for 2 h whilst rotating at RT. Non bound phages were removed in 3 subsequent washing steps with PBS by centrifugation at 500×g for 5 min. After the last washing step cells were resuspended in 1 mg/ml trypsin in PBS and incubated at RT for 30 min. Trypsin inhibitor (2 mg/ml in MQ) was added to neutralize the enzyme and cells were spun down at 500×g for 5 min. Eluted phages were used to infect exponentially growing E. coli TG1 for 30 min at 37° C. Infected bacteria were added to LB medium supplemented with 100 μg ampicillin and 2% glucose and grown 0/N at 37° C. in a shaker. Output of this selection was used in the second round, where phages preblocked with 2% BSA in PBS for 30 min were incubated with 10 nM-10 pM biotynylated HER2-ECD for 2 h at RT in a head-over-head rotor. HER2-ECD was biotinylated with EZ-Link® NHS-Biotin according to the manufacturer's protocol using 5 fold molar excess of biotin (ThermoScientific, Rockford, USA). Non-bound biotin was removed on Zeba Desalt Spin Columns (ThermoScientific, Rockford, USA). Dynabeads® M-270 Streptavidin (Invitrogen Dynal AS, Oslo, Norway) were washed once with PBS, blocked for 30 min at RT with 2% BSA in PBS and then added to the solution of phages and biotynylated HER2-ECD for 1 h at RT. After incubation on a head-over-head rotor, the beads were washed 10 times with 0.05% Tween-20 in PBS and twice in PBS. Bound phages were eluted with trypsin and used to infect E. coli TG1 as described above. In this set up VHH-phage 11A4 was selected based on its ability to bind the HER2 ectodomain with high affinity.

DNA was isolated from bacterial cell cultures of 1C8 and 11A4 clones using the Qiagen Midiprep DNA isolation method (Qiagen, Venlo, The Netherlands).

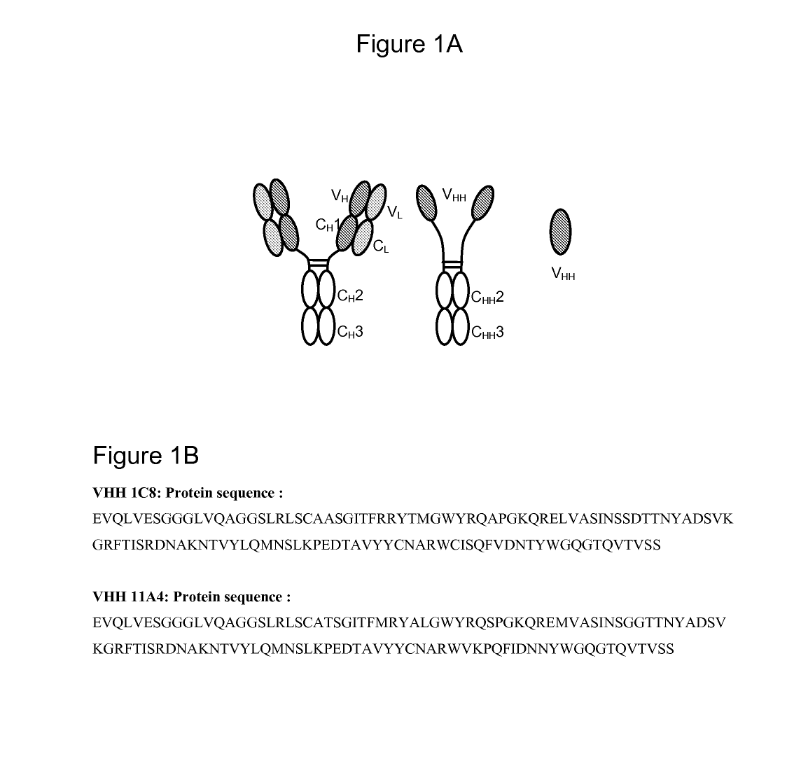

Finally, the coding sequences of VHH 1C8 and 11A4 were identified by performing sequence analysis. The amino acid sequences of VHH 1C8 and 11A4 are depicted in FIG. 1.

### Construction of VHH-Encoding Adapter Constructs.

The construction of the gene encoding the amino-terminal D1 domain of the mCEACAM1a receptor (soR) has been described before (20). In short, a PCR was performed on plasmid pCEP4:sMHVR-Ig (kindly provided by T. Gallagher) with

forward primer
5′-CATGGGCCCAGCCGGCCGAGCTGGCCTCAGCACAT-3′
and
reverse primer
5′-CATGGCGGCCGCGGGGTGTACATGAAATCG-3′.

The resulting DNA fragment soR contained a 5′ SfiI site and a 3′ NotI site (underlined in the primers) and were subsequently cloned with these restriction enzymes into the expression vector pSecTag2, resulting in the expression vector pSTsoR-x-mychis to allow the generation of soR-VHH expression cassettes (20).

To generate the expression cassette VHH-soR, the expression vector pSecTag2 was first provided with a linker containing an additional Hpal site downstream of the NotI site. The soR gene was replaced with a smaller soR, lacking its natural signal sequence, generated by PCR using

forward primer
5′-CAGTGCGGCCGCCGAAGTCACCATTGAGGCTGT-3′
and
5′-ACTGGTTAACGGGGTGTACATGAAATCGC-3′

with a NotI and an Hpal restriction site (underlined) resp. This resulted in the expression vector pST-x-soRmychis.

VHH sequences of 1C8 and 11A4, both directed against human HER2 were obtained by PCR using

forward primer
5′-GCGGCCGCCGAGGTGCAGCTGGTGGAG-3′
and
reverse primer
5′-GCGGCCGCTGAGGAGACGGTGACCTG-3′.

The resulting PCR fragments were digested with NotI (underlined in primers) and subsequently cloned into this restriction site in both expression vectors. The correct orientation and sequence of the insert was confirmed by sequencing.

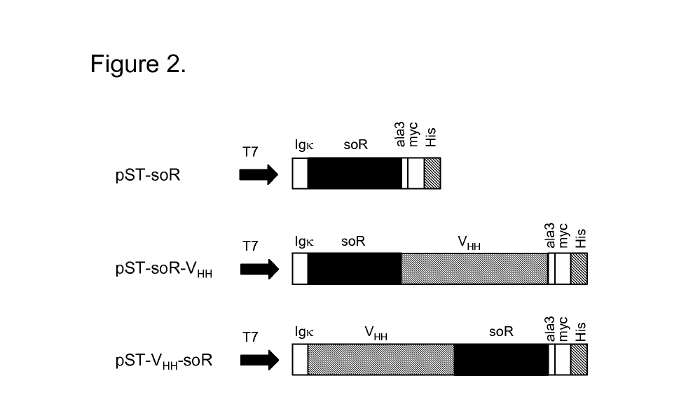

A schematic representation of the constructs is given in FIG. 2. All constructs encode for the N-terminal domain of mCEACAM1a in fusion (either C-terminally for soR-VHH or N-terminally for VHH-soR) with a VHH sequence. They are preceded with an amino-terminal Igκ signal sequence and followed with a carboxy-terminal myc-His tag while under the control of a T7 promoter. Between the soR and VHH fragments a three-Ala linker is present.

### Production of the Adapter Proteins.

For production of the soR-based adapter proteins, subconfluent monolayers of Ost7-1 cells were inoculated at a multiplicity of infection of 5 with the vaccinia virus expressing a T7 polymerase (vTF7-3) (t=0 h) and transfected (t=1 h) with pST-soR, pST-soR-VHH, or pST-VHH-soR using Lipofectin (Life Technologies, Ltd., Paisley, United Kingdom). The medium was refreshed at t=4.5 h, harvested at t=20 h, and centrifuged for 10 min at 3,000 rpm to clear it from cell debris. The supernatants containing the soR proteins were loaded onto a 20% sucrose cushion and centrifuged for 90 min at 13,000 rpm to remove vTF7-3 virus. The protein batches were stored at −20° C.

### Cells and Viruses.

Murine Ost7-1 cells (53) (obtained from B. Moss), hamster CHO-His.scFv (54) (obtained from T. Nakamura), human ovarian cancer cell lines SKOV3 (ATCC HTB-77) and MCF7 (ATCC HTB-22) were all maintained in Dulbecco's modified Eagle's medium (DMEM; Cambrex Bio Science, Verviers, Belgium) containing 10% fetal calf serum (FCS), 100 IU of penicillin/ml, and 100 μg/ml gentamycin (all from Life Technologies, Ltd., Paisley, United Kingdom). Stocks of MHV-A59 were grown and titrated as described before (51).

### Immunoperoxidase Staining of Cell Cultures.

Cells inoculated with MHV in the presence or absence of adapter proteins were fixed with PBS containing 3.7% paraformaldehyde at 20 h post inoculation. The cells were permeabilized with PBS containing 1% Triton X-100, and subsequently incubated with k134 anti-MHV serum diluted 1:300, followed by swine anti-rabbit peroxidase (DAKO, Glostrup, Denmark) diluted 1:300, both in PBS containing 5% fetal bovine serum. The cells were stained with AEC (Brunschwig, Amsterdam, The Netherlands) according to the manufacturer's protocol and analyzed by light microscopy.

### Results

### Functionality of Adapter Proteins.

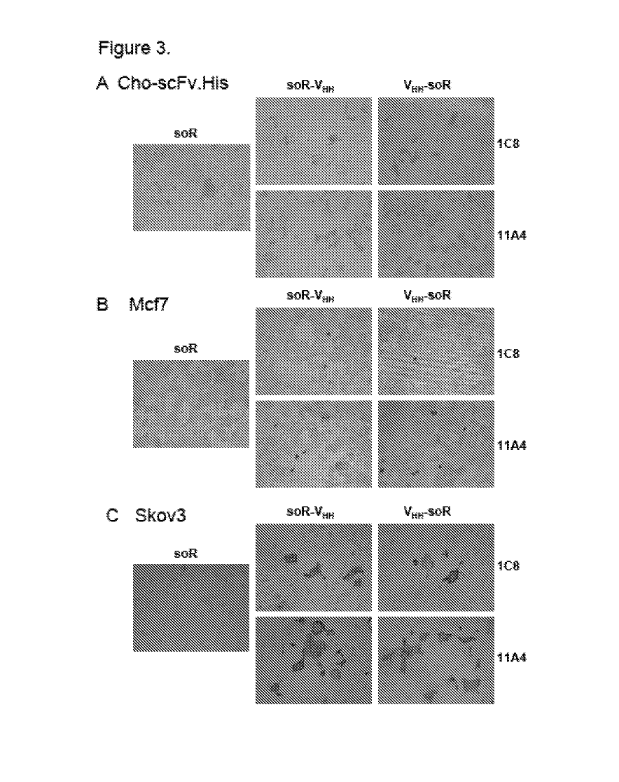

To study the targeting capacities of the soR-VHH adapter proteins, it was first tested whether these proteins were properly produced by testing their ability to infect the control cell line CHO-scFv.His cells (constitutively expressing an artificial His receptor). MHV-A59 was preincubated with soR-1C8, soR-11A4, 1C8-soR, 11A4-soR, or with control supernatant containing soR without targeting device and these mixtures were inoculated in parallel for 2 h onto these cells. At 20 h post inoculation the cells were fixed and immunostaining was performed using a polyclonal antibody directed against MHV. The data show that all adapter proteins were able to redirect MHV to the CHO-His.scFv cells (FIG. 3A), as staining was observed in all cases. In addition, syncytia formation could be observed, a hallmark of a coronavirus infection. Cells inoculated with cell culture supernatant from mock-transfected cells remained negative as expected (not shown). Interestingly, both the soR-VHH and VHH-soR were able to redirect MHV, indicating that both an N-terminal and C-terminal extension of the VHH was tolerated and not detrimental for its targeting ability.

### Soluble Receptor-Mediated MHV Infection of Cells Expressing Human HER2.

Next, a similar targeting experiment was performed in which HER2-expressing human ovarian carcinoma cells were inoculated with MHV-adapter protein mixtures. To this end, both MCF7 and SKOV3 cells, with low and high HER2 expression, respectively were used. At 20 h post infection, immunostaining of the inoculated cells showed that both cell lines could be infected with MHV redirected with the VHH-containing adapter proteins, but not with the control protein (FIGS. 3B and 3C). The number of infected MCF7 cells was considerably less than the number of positive SKOV3 cells, likely due to the differences in HER2-receptor expression by these cells. In addition, clear syncytia formation could be observed for SKOV3, but not for MCF7 cells. In conclusion, the infection was dependent on both the targeting moiety represented by the specific VHH as well as on the HER2 receptor expression.

### Example 2

### Construction of the Vectors for Targeted Recombination

To allow expression of the adapter proteins from an additional expression cassette in the viral genome of MHV-A59, the genes encoding VHH-soRmychis and soR-VHH-mychis, including an upstream transcription regulation sequence (TRS) (20), are first cloned into pXH1802 (48), containing approximately 1,200 bp of the 3′ end of the replicase gene 1b fused to the S gene of MHV-A59. To this end, the inserts are obtained by digestion of the vectors with EcoRV and PmeI, and the purified fragments are cloned into the Klenow-treated HindIII site of pXH1802. Then, the resulting plasmids are digested with RsrII and AvrII and the obtained fragments are cloned into pMH54 (16), treated with the same enzymes. This resulted in the transcription vectors pMH-soR-VHH-mycHis and pMH-VHH-soR-mycHis, suitable for targeted recombination.

### Targeted Recombination.

The adapter genes VHH-soR-mycHis and soR-VHH-mycHis are introduced as additional expression cassettes into the MHV genome by targeted RNA recombination as described previously (48, 16, 49, 20). Briefly, donor RNAs transcribed in vitro from PacI-linearized plasmids pMH-VHH-soR-mycHis, and pMH-soR-VHH-mycHis are transfected by electroporation into feline FCWF-4 cells that had been infected with fMHV at a multiplicity of infection (MOI) of 0.5 4 h earlier. These cells are then plated in culture flasks, and the culture supernatant is harvested 24 h later. Progeny virus is plaque purified, and virus stocks are grown on LR7 cells. After confirmation of the presence of the additional expression cassettes by reverse transcription (RT)-PCR with purified viral RNA from these virus stocks, the virus titers of the stocks are determined by endpoint dilution on LR7 cells. These passage 2 virus stocks are subsequently used in the experiments. For each virus, two independent recombinants are generated as a control for effects caused by unintended mutations in other parts of the viral genome.

Viral RNA Isolation and RT-PCR.

First, from 140 μl virus-containing culture supernatant, viral RNA is isolated using a QIAGEN viral RNA isolation kit (according to the manufacturer). Reverse transcription with the isolated RNA is then performed using reverse primer 1127 (5′-CCAGTAAGCAATAATGTGG-3′), located at nt 24,110 to 24,128 of the MHV genome (GenBank accession no. NC 001846). PCR is performed using primers 1173 (5′-GACTTAGTCCTCTCCTTGATTG-3′, nt 21650 to 21671) and 1260 (5′-CTTCAACGGTCTCAGTGC-3′, nt 24,041 to 24,058), overlapping the region that contains the inserted expression cassette. The resulting fragments are subsequently sequenced to confirm the sequence of the inserts.

Inoculation of target cells. An amount of 1×10⁵ CHO-His.scFv, SKOV3 and MCF7 cells are inoculated with 0.5×10⁵ TCID₅₀. At 16 h p.i., the cells are fixed and immunoperoxidase staining using MHV antiserum (as described above) is performed to analyze whether the cells express viral proteins, thus became infected with recombinant MHV.

### LITERATURE

1) Cervantes-Garcia, D., R. Ortiz-Lopez, N. Mayek-Perez, and A. Rojas-Martinez. Ann. Hepatol. 7:34-45 (2008).
2) Guo, Z. S., S. H. Thorne, and D. L. Bartlett. Biochim. Biophys. Acta 1785:217-231 (2008).
3) Liu, T. C., and D. Kim. Gene Ther. 15:877-884 (2008).
4) Russell, S. J., and K. W. Peng. Trends Pharmacol. Sci. 28:326-333 (2007).
5) Waehler, R., S. J. Russell, and D. T. Curiel. Nat. Rev. Genet. 8:573-587 (2007).
6) Galanis, E., R. Vile, and S. J. Russell. Crit. Rev. Oncol. Hematol. 38:177-192 (2001).
7) Ring, C. J., J. Gen. Virol. 83:491-502 (2002).
8) Wickham, T. J., Nat. Med. 9:135-139 (2003).
9) Holmes K. V. & Lai M. M. The viruses and their replication. In: Howley P M (ed). Fields Virology Lippincott-Raven Publishers: Philadelphia 1996; pp 1075-1093.
10) Cavanagh D. The coronavirus surface protein. In: Siddell S G (ed). The Coronaviridae Plenum Press: New York, N.Y. 1995; pp 73-103.
11) Vennema H. et al., J. Virol. 64; 339-346 (1990).
12) Benbacer L. et al., J. Virol. 71; 734-737 (1997).
13) Wentworth D. E. & Holmes K. V., J. Virol. 75; 9741-9752 (2001).
14) Compton S. R. et al., J Virol; 66; 7420-7428 (1992).
15) Tresnan D. B., Levis R. & Holmes K. V., J. Virol.; 70:8669-8674 (1996).
16) Kuo L. et al., J. Virol. 74; 1393-1406 (2000).
17) Haijema B. J., Volders H. & Rottier P. J., J Virol; 77; 4528-4538 (2003).
18) Yount, B. et al., J. Virol. 76; 11065-11078 (2002)
19) Dveksler G S et al., J. Virol. 67; 1-8 (1993).
20) Verheije, M. H., T. Wurdinger, V. W. van Beusechem, C. A. de Haan, W. R. Gerritsen, and P. J. Rottier, J. Virol. 80; 1250-1260. (2006)
21) Wurdinger, T., M. H. Verheije, K. Broen, B. J. Bosch, B. J. Haijema, C. A. de Haan, V. W. van Beusechem, P. J. Rottier, and W. R. Gerritsen, J. Virol. 79; 15314-15322 (2005).
22) Wurdinger, T., M. H. Verheije, M. Raaben, B. J. Bosch, C. A. de Haan, V. W. van Beusechem, P. J. Rottier, and W. R. Gerritsen. Gene Ther. 12; 1394-1404 (2005).
23) Verheije, M. H. et al., Journ. Virology 83; 1250-1260 (2009),
24) Winter, G. et al., Annu. Rev. Immunol. 12; 433-455 (1994).
25) Hoogenboom, H. R. et al, Immunotechnology 4; 309-318 (1998))
26) Gharhoudi, M. A. et al., FEBS Letters 414; 521-526 (1997))
27) Lauwerijs, M, et al., EMBO J. 17; 3512-3520 (1998),
28) Cortez-Retamozo, V. et al., Cancer Res. 64; 2853-2857 (2004))
29) Frenken L. et al., Res. Immunol. 149; 589-598 (1998)).
30) Revets, H. et al., Exp. Opin. Biol. Ther. 5; 111-124 (2005)
31) Harmsen, M. M. and De Haard, H. J., Appl. Microbiol. Biotechnol. 77; 13-22 (2007)
32) Vanlanschoot, P. et al., Antiviral Res. 92; 389-407 (2011)
33) Wesolowski, J. et al., Med. Microbiol. Immunol. 198; 157-174 (2009).
34) Vincke, C. et al., J. Biol Chem. 284; 3273-3284 (2008)
35) Boqi Liu et al, J. Biotechnol 141; 91-96 (2009)
36) Ren, X. et al., J. Biotechnol 150; 202-206 (2010)
37) Tresnan, D. B and Holmes, K. V., Adv. Exp. Med. Biol. 440; 69-75 (1998)
38) (Seed, B. et al., Nature 329; 840-842 (1987)
39) Fynan, E. F. et al., PNAS 90; 11478-11482 (1993)
40) Ulmer, J. B. et al., Science 259; 1745-1748, (1993)
41) Gorman, C. M. et al., PNAS 79; 6777-6781 (1982)
42) Stacey et al., J. Virology 50; 725-732 (1984)
43) Sprague J. et al., J. Virology 45; 773 (1983)
44) Berman, P. W. et al., Science 222; 524-527, (1983)
45) Brinster, R. L. et al., Nature 296; 39-42, (1982)
46) Voellmy et al., Proc. Natl. Acad. Sci. USA, 82; 4949-53 (1985)
47) Tang et al., Nature 356; 152-154 (1992)
48) de Haan, C. A., P. S. Masters, X. Shen, S. Weiss, and P. J. Rottier. Virology. 296: 177-89 (2002).
49) Verheije, M. H., M. L. Lamfers, T. Wurdinger, G. C. Grinwis, W. R. Gerritsen, V. W. van Beusechem, and P. J. Rottier. J Virol. 83:7507-16 (2009).
50) Chomczynski, P., and N. Sacchi., Anal. Biochem. 162: 156-159 (1987).
51) de Haan, C. A., L. van Genne, J. N. Stoop, H. Volders, and P. J. Rottier. J Virol. 77: 11312-11323 (2003)
52) De Haard, H. J., S. Bezemer, A. M. Ledeboer, W. H. Muller, P. J. Boender, S. Moineau, M. C. Coppelmans, A. J. Verkleij, L. G. Frenken, and C. T. Verrips., J. Bacteriol. 187:4531-4541 (2005)
53) Fuerst, T. R., E. G. Niles, F. W. Studier, and B. Moss., Proc Natl Acad Sci USA. 83; 8122-8126 (1986).
54) Kaufmann, M., P. Lindner, A. Honegger, K. Blank, M. Tschopp, G. Capitani, A. Pluckthun, and M. G. Grutter., J Mol Biol. 318; 135-47 (2002)

1-8. (canceled)

9. A recombinant Coronavirus comprising an expression cassette that comprises an RNA or DNA molecule comprising a nucleotide sequence encoding a bi-specific adapter;
wherein said bi-specific adapter is a protein that comprises a Coronavirus Spike-protein receptor moiety and a camelid VHH antibody moiety; and
wherein said nucleotide sequence is under the control of a transcription regulatory sequence (TRS).

10. The recombinant Coronavirus of claim 9, wherein said Coronavirus Spike-protein receptor moiety comprises only a soluble part of the Coronavirus Spike-protein receptor.

11. The recombinant Coronavirus of claim 10, wherein said Coronavirus Spike-protein receptor is selected from the group consisting of Murine Hepatitis Virus (MHV) Spike-protein receptor, Feline Infectious Peritonitis Virus (FIPV) Spike-protein receptor, and Transmissible Gastroenteritis Virus (TGEV) Spike-protein receptor.

12. The recombinant Coronavirus of claim 9, wherein said Coronavirus Spike-protein receptor is selected from the group consisting of Murine Hepatitis Virus (MHV) Spike-protein receptor, Feline Infectious Peritonitis Virus (FIPV) Spike-protein receptor, and Transmissible Gastroenteritis Virus (TGEV) Spike-protein receptor.

13. The recombinant Coronavirus of claim 12, wherein said camelid VHH antibody moiety is directed against a tumor-specific antigen.

14. The recombinant Coronavirus of claim 11, wherein said camelid VHH antibody moiety is directed against a tumor-specific antigen.

15. The recombinant Coronavirus of claim 10, wherein said camelid VHH antibody moiety is directed against a tumor-specific antigen.

16. The recombinant Coronavirus of claim 9, wherein said camelid VHH antibody moiety is directed against a tumor-specific antigen.

17. Pharmaceutical composition comprising the recombinant Coronavirus of claim 16 and a pharmaceutically acceptable solution.

18. A pharmaceutical composition comprising the recombinant Coronavirus of claim 15 and a pharmaceutically acceptable solution.

19. A pharmaceutical composition comprising the recombinant Coronavirus of claim 14 and a pharmaceutically acceptable solution.

20. A pharmaceutical composition comprising the recombinant Coronavirus of claim 13 and a pharmaceutically acceptable solution.

21. A pharmaceutical composition comprising the recombinant Coronavirus of claim 12 and a pharmaceutically acceptable solution.

22. A pharmaceutical composition comprising the recombinant Coronavirus of claim 11 and a pharmaceutically acceptable solution.

23. A pharmaceutical composition comprising the recombinant Coronavirus of claim 10 and a pharmaceutically acceptable solution.

24. A pharmaceutical composition comprising the recombinant Coronavirus of claim 9 and a pharmaceutically acceptable solution.

25. A method of treating a tumor in an animal that has a tumor comprising administering the pharmaceutical composition of claim 24 to said animal.

25. A method of treating a tumor in an animal that has a tumor comprising administering the pharmaceutical composition of claim 23 to said animal.

26. A method of treating a tumor in an animal that has a tumor comprising administering the pharmaceutical composition of claim 21 to said animal.

27. A method of treating a tumor in an animal that has a tumor comprising administering the pharmaceutical composition of claim 17 to said animal.
